(19) United States
(12) Patent Application Publication
(10) Pub. No.: US 2023/0135572 A1
(43) Pub. Date: May 4, 2023

(54) LIGHT APPARATUS FOR USE WITH MEDICAL PROBE DEVICE

(71) Applicant: Tova Neuhauser, Monsey, NY (US)

(72) Inventor: Tova Neuhauser, Monsey, NY (US)

(21) Appl. No.: 18/050,727

(22) Filed: Oct. 28, 2022

Related U.S. Application Data
(60) Provisional application No. 63/273,631, filed on Oct. 29, 2021.

(51) Int. Cl. A61B 90/35

(52) U.S. Cl. CPC A61B 90/35

(57) ABSTRACT

The present invention generally relates to a light apparatus for use with medical probe devices and a method of use thereof. Specifically, the invention relates to a lighting apparatus configured to illuminate medical or operating fields when medical probe devices, for example, ultrasound probes, are introduced onto or into organs or body lumens during various procedures, and methods of use thereof.

### CROSS-REFERENCE TO RELATED APPLICATIONS

This application claims of the benefit of Provisional Application No. 63/273,631 entitled LIGHT APPARATUS FOR USE WITH MEDICAL PROBE DEVICE filed on Oct. 29, 2021, the entire disclosure of which is incorporated herein by reference.

### FIELD OF THE INVENTION

The present invention generally relates to a light apparatus for use with medical probe devices and a method of use thereof. Specifically, the invention relates to a lighting apparatus configured to illuminate medical or operating fields when medical probe devices, for example, ultrasound tranducers or probes, are introduced onto or into organs or body lumens during various procedures, and methods of use thereof.

### BACKGROUND

Many medical devices have been developed for intracorporeal and extracorporeal use. In some scenarios, imaging or visualization devices are used to facilitate navigation and examination through and of the anatomy of a patient. A variety of imaging or visualization medical devices such as diagnostic probes and the like have been developed, having their advantages and disadvantages. Traditional ultrasound probes and other visualization devices, for example, ultrasound transvaginal or endovaginal probes, oftentimes lack sufficient visualization during various procedures due to, for example, a lack of proper lighting. The inability to view where a probe or similar visualization or imaging device is being placed, or its particular location, can cause patients to experience unnecessary discomfort during various procedures requiring the insertion of probes and similar visualization or imaging devices into certain body parts, for example, during introduction to or examination of a patient vaginal canal or cervix.

Certain imaging procedures, for example, vaginal ultrasound imaging procedures, must be performed in dark rooms for optimal visualization of contrasts in the ultrasound imaging. The options for medical professionals performing such procedures are currently, in the case of vaginal ultrasounds, (i) inserting a transvaginal or endovaginal probe in a dark or dimly lit room without proper visualization during the probe insertion process and subsequently performing the ultrasound, (ii) inserting the probe in a lit room for proper visualization during the probe insertion process and having a third party power off the lights so that the ultrasound be performed under optimal conditions in the dark or dimly lit room, or (iii) inserting the probe in a lit room and subsequently performing the ultrasound under sub-optimal conditions in the lit room.

Therefore, there is a need in the art for a medical device comprising a lighting apparatus configured to selectively illuminate a medical field. In some scenarios, the lighting apparatus may comprise one or more lighting modules. In some examples, the lighting apparatus may be integrally formed on a medical imaging or visualization probe or may be an add-on or accessory configured to attach to prior-developed probes (e.g., retrofitted to prior-developed probes), to facilitate increased illumination and visualization of a medical field during use of a selected probe. These and other features and advantages of the present invention will be explained and will become obvious to one skilled in the art through the summary of the invention that follows.

### SUMMARY OF THE INVENTION

The present invention is a medical device and methods of manufacture and use thereof. In some embodiments, the medical device may comprise a lighting apparatus comprising a body member having one or more lighting modules. In some examples, the body member may be engageable with an outer portion of a medical device such as a transducer, probe, endoscope, or any other similarly suitable visualization or imaging device. In some embodiments, the lighting apparatus may be integrally formed with a visualization or imaging medical device such as a probe, endoscope or other visualization or imaging device.

### BRIEF DESCRIPTION OF THE DRAWINGS

Accompanying this written specification is a collection of drawings of exemplary embodiments of the present invention. One of ordinary skill in the art would appreciate that these are merely exemplary embodiments, and additional and alternative embodiments may exist and still be within the spirit of the invention as described herein.

FIG. 1 shows an elevational view of a light apparatus for use with medical probe devices integrally formed with an exemplary probe device, in accordance with an embodiment of the present invention.

FIG. 2 shows an elevational view of a light apparatus for use with medical probe devices configured as an accessory engaged with an exemplary probe device, in accordance with an embodiment of the present invention.

FIG. 3 shows a perspective view of a light apparatus for use with medical probe devices, in accordance with an embodiment of the present invention.

### DETAILED SPECIFICATION

The present invention generally relates to a medical device light apparatus which may comprise a substantially tubular body member formed with one or more lighting modules and having one or more interior walls engageable with an outer portion of an ultrasound probe or the like medical device. In some scenarios, the lighting apparatus may be formed on an ultrasound probe or like medical device.

It is an aspect of the present invention to provide a lighting apparatus, integrally formed with or configured to couple to probe devices having visualization or imaging capabilities. For example, the lighting apparatus may be formed on or couple with ultrasound probes to facilitate placement or insertion of a probe, for example, a vaginal probe, accurately into position in a dark or dimly lit room. In some scenarios, use of the lighting apparatus in conjunction with the probe may avoid the need for a medical professional (e.g., a nurse, doctor, technician, etc.) to place or introduce a medical probe on or into a patient in a dark room, without proper visualization. In some scenarios, use of the lighting apparatus in conjunction with the probe may avoid the need for a medical professional to have to, subsequently to appropriately placing or introducing a medical probe on or into a patient in a well-lit exam room, shut off the light to adequately perform a selected ultrasound. In some scenarios, use of the lighting apparatus in conjunction with the probe may avoid the need for a medical professional to have to perform an ultrasound under sub-optimal conditions (e.g., in a room with the lights on) because proper lighting was needed to place or introduce the probe onto or into a patient body.

Embodiments of the present invention are generally directed at a lighting apparatus configured to provide lighting during various medical and/or diagnostic procedures which may need to be performed in a dark room. For example, during ultrasound imaging procedures, an optimal exam room should be dark or dimly lit. The present invention may provide a localized or generalized light source on the medical tool, for example, the ultrasound probe, to support utilization of the medical tool in a dark or dimly lit room.

In accordance with embodiments of the present invention, the lighting apparatus may be integrally formed on an ultrasound probe or similar medical device or may be configured to attach to an existing ultrasound probe or similar medical device. In some embodiments, the lighting apparatus may comprise a body member which may be formed of a substantially pliable and elastic material or a substantially rigid material.

In accordance with embodiments of the present invention, the body member may be formed of a substantially rigid and/or nonflexible material, for example, a plastic, metal, vinyl (e.g., rigid polyvinyl chloride (PVC)) or any other similar substantially rigid and/or nonflexible material or any combination thereof. In some examples, the body member may be formed of a substantially flexible and/or elastic material, for example, the body member may be configured as a strap or band which may be formed of a substantially flexible and/or elastic material, for example, a silicone, elastomer, flexible PVC or any other similar substantially flexible and/or elastic material or any combination thereof. In some embodiments, the body member may be formed of a combination of a rigid, nonflexible material and a flexible, elastic material.

In examples where the lighting apparatus is configured to attach to (e.g., retrofit) an existing ultrasound probe or similar medical device, the body member may have an interior portion comprising one or more sidewalls adapted to facilitate engagement with medical probes, for example, by being configured to abut at least a portion of the exterior of pre-existing medical probes, for example, endovaginal probes known in the art. In some examples, the body member may comprise an opening with a substantially round interior portion forming an interior chamber which may be configured to facilitate engagement with ultrasound probes and/or similar medical devices known in the art, which may be utilized for various intracorporeal or extracorporeal procedures, for example, by being adapted to abut at least a portion of the exterior of such probes. In such scenarios, the size (e.g., circumference) of the body member opening may be of a fixed and/or predetermined size, for example, corresponding in size (e.g., circumference) to the exterior of a medical probe or other device. In some scenarios, the size of the body member opening may be adjustable (e.g., elastic) to support its engagement with medical probes and/or device of varying sizes and/or attachment of the body member to different positions on a given medical device.

In examples where the lighting apparatus is integrally formed with an ultrasound probe or similar medical device, the lighting apparatus may be configured to attach to the device at any location on the device, for example, on the handle of a medical probe, between the handle and the shaft of the probe, or on the probe shaft itself.

In accordance with embodiments of the present invention, the body member of the lighting apparatus may be configured to attach to a medical or diagnostic probe utilizing any suitable attachment means including by means of a friction or snap fit with the probe, or the use of any suitable attachment means or adhesives having the ability to be sterilized or disinfected (e.g., autoclaved or soaked in disinfectant) and which may sufficiently secure the body member to a selected medical probe or similar device. In any embodiment, the attachment means may be releasable or permanent attachment means.

In accordance with embodiments of the present invention, the body member of the lighting apparatus may be configured to attach to a medical or diagnostic probe at any location on the medical probe, for example, on the handle of the probe, between the handle and the shaft of the probe, or on the probe shaft itself. In some examples, the body member of the lighting apparatus may be configured to function like a bracelet, and may be configured to slide along a probe in an open configuration until a selected position is reached, and subsequently closed or secured to the handle at the selected position. In some examples, the body member may include a rotating element, for example, a hinge such as a butterfly hinge or strap hinge, and a locking mechanism, for example, a clasp, hasp lock, latch lock, hook and loop lock or any other similarly suitable locking mechanism. In some scenarios, the hinge may operably or rotatably connect a first end of a first body member section to a first end of a second body member section and the locking mechanism may releasably connect a second end of the first body member section to a second end of the second body member section. In some embodiments, the hinge may substantially oppose the locking mechanism (e.g., the clasp locking mechanism). In some examples, the body member may be moved from a locked position wherein the locking mechanism or clasp secures the second end of the first body section to the second end of the second body section, to an unlocked position wherein the locking mechanism is released (e.g., opened), and the second end of the first body section is disconnected or released from the second end of the first body section. In some examples, the lighting apparatus may be attached or secured to a probe by placing an unlocked body member on the probe and using the locking mechanism to secure the body member to the probe. The position of the lighting apparatus on a given probe may depend on the intended use of the lighting apparatus, the medical procedure being performed, or the medical probe being utilized.

In accordance with embodiments of the present invention, the lighting apparatus may comprise a body member having one or more lighting modules configured to illuminate at least a portion of a medical probe, for example, to support visualization of at least a portion of the medical probe during use of the medical probe in a dark or dimly lit exam room. In some embodiments, the one or more lighting modules may be configured to illuminate at least a portion of a region around a medical probe, for example, to support accurate positioning of the medical probe during use of the medical probe in a dark or dimly lit room.

In accordance with embodiments of the present invention, the lighting modules of the lighting apparatus may be an array of light-emitting diodes (“LEDs”) or other similarly suitable light bulbs that may be individually or collectively logically addressable. In some embodiments, the lighting apparatus may comprise a circuit board, for example a printed circuit board (PCB), formed of a substrate such as polyimide film, having conductive paths and a plurality of LEDs installed thereon. In some embodiments, the lighting apparatus may include a control circuit unit for controlling the color, intensity and timing of LEDs. In any embodiment, the lighting modules may be configured to be visible under a variety of lighting conditions, from dark conditions, such as a darkened or dimly lit rooms or areas to bright conditions, such as a brightly lit indoor rooms or areas.

In accordance with embodiments of the present invention, within the body member of the lighting apparatus may be a lighting unit comprising of at least one or more LEDs or other light sources, with or without a printed circuit board (PCB), and a battery or other power source and a switch to control the illumination. The light source, with or without the PCB, and the battery or other power source may be in communication with circuitry as to provide illumination to the lighting module(s) of the apparatus.

In accordance with embodiments of the present invention, the function of the lighting apparatus, for example, the lighting modules of the lighting apparatus, may be controlled by a microcontroller unit (MCU), which may draw power from a power source. The power source may be a direct current (DC) power source. In some embodiments, the power source may be batteries, such as rechargeable batteries. For example, the power source may be lithium-ion (Li-ion) batteries or lithium polymer (Li-poly) batteries. However, in embodiments where the power source are batteries, the batteries may be other suitable types (e.g., lead acid, nickel cadmium, nickel metal hydride). In some scenarios, one or more batteries or other power sources or a combination of different types and sizes of batteries or other power sources may be used for brighter or longer illumination. In some embodiments, the power source may be a higher voltage alternating current (AC) source, coupled with a transformer, to bring the supplied power to acceptable DC voltage and current levels. In some embodiments, the power source may be capacitors.

Furthermore, the size and placement of the batteries or other power sources can also differ depending, for example, on the desired placement of the lighting apparatus. For example, a bigger power source may be used on larger medical devices to provide more electrical energy to power more light sources at a higher illumination or for a longer period of time. For placement of the apparatus on smaller medical devices, for example, the batteries or other power sources can be smaller to accommodate the availability of space.

In accordance with embodiments of the present invention, a controlling mechanism (e.g., a “controller”) may be disposed on or embedded within the body member to allow for the control of the color and/or intensity and/or powering on/off of the illumination. The controlling mechanism may be a button, a switch, a touch sensor, wireless receiver, etc. The control may also be a predetermined set of colors and/or intensity of illumination or it can be a sliding scale of colors and/or intensity of illumination. In some scenarios, the power source may be directed to be turned on or off by the controlling mechanism (e.g., a switch or similar button capable of starting or stopping to supply power to the power source). In accordance with embodiments of the present invention, the lighting apparatus may be powered on together with or separately from the medical probe.

In accordance with embodiments of the present invention, the controlling mechanism may be configured to direct the lighting modules of the lighting apparatus. In some examples, the controlling mechanism may be external to the body member of the lighting apparatus and may communicate with a transceiver unit through a wireless or physical communication protocol, such as Bluetooth®, WiFi®, USB and similar wireless or physical communication protocols. In some embodiments, the controlling mechanism may be located on the lighting apparatus and may communicate with the lighting apparatus through electrical or wired communication protocols. In some embodiments, the controlling mechanism is a piece of hardware dedicated to the controlling of the lighting modules, or the lighting apparatus as a whole. The controlling mechanism may be any computing device having the ability to communicate with one or more lighting modules.

In accordance with embodiments of the present invention, the MCU may communicate with a plurality of light drivers, each of which may control at least one light. In some embodiments, the lights may be controlled by light drivers having a number of output lines. In some embodiments, transistors may regulate the power sent to the lights.

In accordance with embodiments of the present invention, the body member may be made of any durable material, including, for example, a metal, plastic, vinyl or any other similarly suitable durable material or any combination thereof. In some embodiments, the durable material may be scratch resistant.

In accordance with embodiments of the present invention, a medical device may comprise a visualization means and a lighting means disposed in a body member comprising one or more lighting modules, a portion of each lighting module at least partially disposed within the body member. In some examples, the body member may be integrally formed on a handle of the medical device. In some embodiments, the visualization means may be an ultrasound imaging means. In some examples, the lighting means may comprise one or more lighting modules, each comprising one or more lights coupled to one or more light drivers.

In accordance with embodiments of the present invention, a lighting apparatus for use with medical probes may comprise a body member formed as a housing and one or more lighting modules, each comprising a light coupled to a a light driver, at least a portion of each lighting module disposed in the housing. In some examples, a control unit may be coupled to the body member, the control unit comprising a power source, a communication transceiver, at least one of the light drivers coupled to the communication transceiver and adapted to control at least one of the lights. In some examples, the body member may be specifically configured to attach to a medical probe. In some embodiments, the body member may comprise a rotating element and a locking mechanism. In some examples, the rotating element may oppose the locking mechanism. In some embodiments, the locking mechanism may be selected from the group consisting of: a clasp, a hasp lock, and a latch lock. In some examples, the rotating element may be a hinge. In some examples, a portion of the hinge may rotate to open the body member by moving a first body member section away from a second body member section to provide access to an interior portion of the body member. In some scenarios, a portion of the body member may be movable about a hinge to support movement of the body member from a closed position to an open position. In some examples, the body member may be formed with an opening extending towards a channel to form an internal chamber having one or more walls adapted to abut at least a portion of a medical probe.

In accordance with embodiments of the present invention, a lighting apparatus for releasable attachment to a medical probe may comprise a body member that is a frame with an inner surface and an outer surface, wherein the inner surface defines a cavity that passes through the body member and is shaped to abut and selectively engage an exterior surface of the medical probe, and one or more lighting modules, a portion of each lighting module at least partially disposed within the body member. In some embodiments, the lighting apparatus may further comprise a control unit in communication with the lighting modules, the control unit comprising a power source, a communication transceiver, and one or more light drivers coupled to said communication transceiver and adapted to control at least one of said lights. In some examples, the frame may have a first end and a second end that extend towards each other around the medical probe to define a gap between the first end and the second end of the frame. In some examples, the frame may be continuous and may be configured to wrap around the circumference of an ultrasound probe handle. In some embodiments, the lighting apparatus may be an accessory for a medical probe.

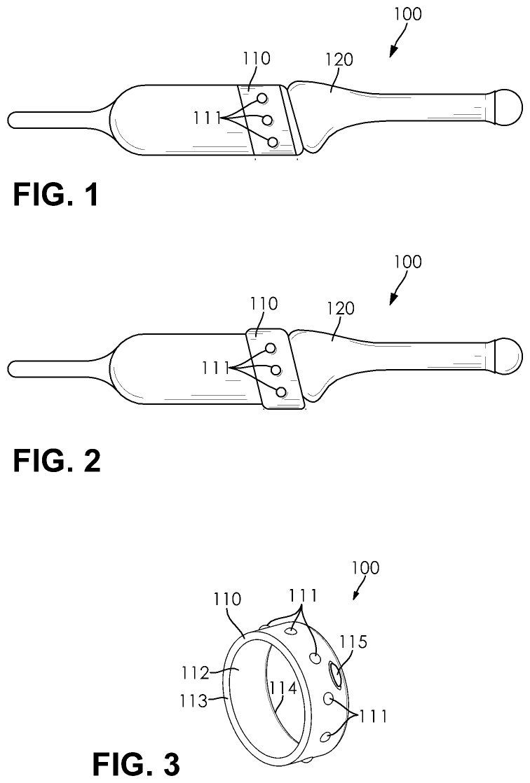

Turning to FIGS. 1-3, a lighting apparatus, in accordance with embodiments of the present invention, is shown.

FIG. 1 shows an elevational view of a light apparatus for use with medical probe devices integrally formed with an exemplary probe device, in accordance with an embodiment of the present invention. As shown in FIG. 1, the lighting apparatus 100 may comprise a body member 110 having one or more light modules 111 disposed thereon. As shown in the illustrated example, the body member 110 may be disposed on a handle of a probe device 120. In any embodiment, the body member 110 of the lighting apparatus may be disposed at any location on the probe device 120, depending on the intended use of the probe device 120 or the lighting apparatus 110.

FIG. 2 shows an elevational view of a light apparatus for use with medical probe devices configured as an accessory engaged with an exemplary probe device 120, in accordance with an embodiment of the present invention. As shown in the depicted example, the lighting apparatus 100 may be configured as an accessory, adapted to engage with a probe device 120. For example, the body member 110 of the lighting apparatus 100 may be configured to engage with a handle or wand of a probe device 120. In any embodiment, the body member 110 of the lighting apparatus may be configured to engage with any portion of the probe device 120.

FIG. 3 shows a perspective view of a light apparatus for use with medical probe devices, in accordance with an embodiment of the present invention. As shown in FIG. 3, the body member 110 may comprise one or more lighting modules 111 and may be formed as a continuous and substantially hollow tubular body, having a substantially tubular interior chamber 112 with a first opening 113 extending towards a second opening 114. In the depicted embodiment, the body member 110 is substantially circular, however, in any embodiment, the body member 110 may be formed in any suitable shape, and may be configured to attach to a probe device 120 using any suitable attachment means, including releasable and permanent attachment means, for example, releasable or permanent adhesives, hook and loop fasteners, or screws. In some examples, the interior chamber 112 of the body member 110 may be semi-circular or substantially “C” shaped (e.g., substantially round, but not continuous). In some embodiments, the body member 110 may be substantially square, rectangular, triangular, or any other geometric shape. As shown in the depicted example, the body member 110 may be provided with a power switch or button 115 to power on the lighting apparatus 100. In any embodiment, any similar actuation member may be provided to power the device on or off.

In accordance with embodiments of the present invention, the lighting apparatus 100 of the present disclosure may facilitate visualization of at least a portion of the medical probe 120 during use of the medical probe in a dark or dimly lit exam room and/or may illuminate at least a portion of a region around a medical probe device 120, for example, to support accurate positioning of the medical probe during use of the medical probe in a dark or dimly lit room. In any embodiment, the lighting apparatus of the present disclosure may support a user's (e.g., doctor, technician, nurse, or other medical professional) visualization during various intracorporeal or extracorporeal procedures, notwithstanding the lighting conditions of the procedure room. Furthermore, in any embodiment, the present invention may provide either or both of focused or generalized lighting to a selected field of operation, which may depend on the intended use of the probe device 120 or the lighting apparatus 100.

The terminology used herein is for the purpose of describing particular embodiments only and is not intended to be limiting of the invention. As used herein, the term “and/or” includes any and all combinations of one or more of the associated listed items. It will further be understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A medical device, comprising:
a visualization means; and
a lighting means disposed in a body member comprising one or more lighting modules, a portion of each lighting module at least partially disposed within the body member.

2. The medical device of claim 1, wherein the body member is integrally formed on a handle of the medical device.

3. The medical device of claim 1, wherein the visualization means is an ultrasound imaging means.

4. The medical device of claim 1, wherein the lighting means comprises one or more lighting modules, each comprising one or more lights coupled to one or more light drivers.

5. A lighting apparatus for use with medical probes, comprising:
a body member formed as a housing; and
one or more lighting modules, each comprising a light coupled to a light driver, at least a portion of each lighting module disposed in the housing.

6. The lighting apparatus for use with medical probes of claim 5, wherein a control unit is coupled to the body member, the control unit comprising a power source, a communication transceiver, at least one of said light drivers coupled to said communication transceiver and adapted to control at least one of said lights.

7. The lighting apparatus for use with medical probes of claim 5, wherein the body member is specifically configured to attach to a medical probe.

8. The lighting apparatus for use with medical probes of claim 5, wherein the body member comprises a rotating element and a locking mechanism.

9. The lighting apparatus for us with medical probes of claim 8, wherein the rotating element opposes the locking mechanism.

10. The lighting apparatus for use with medical probes of claim 9, wherein the locking mechanism is selected from the group consisting of: a clasp, a hasp lock, and a latch lock.

11. The lighting apparatus for use with medical probes of claim 8, wherein the rotating element is a hinge.

12. The lighting apparatus for use with medical probes of claim 11, wherein a portion of the hinge rotates to open the body member by moving a first body member section away from a second body member section to provide access to an interior portion of the body member.

13. The lighting apparatus for use with medical probes of claim 5, wherein a portion of the body member is movable about a hinge to support movement of the body member from a closed position to an open position.

14. The lighting apparatus for use with medical probes of claim 5, wherein the body member is formed with an opening extending towards a channel to form an internal chamber having one or more walls adapted to abut at least a portion of a medical probe.

15. A lighting apparatus for releasable attachment to a medical probe, comprising:
a body member that is a frame with an inner surface and an outer surface, wherein the inner surface defines a cavity that passes through the body member and is shaped to abut and selectively engage an exterior surface of the medical probe; and
one or more lighting modules, a portion of each lighting module at least partially disposed within the body member.

16. The lighting apparatus for releasable attachment to a medical probe of claim 15, further comprising a control unit in communication with the lighting modules, the control unit comprising a power source, a communication transceiver, and one or more light drivers coupled to said communication transceiver and adapted to control at least one of said lights.

17. The lighting apparatus for releasable attachment to a medical probe of claim 15, wherein the frame has a first end and a second end that extend towards each other around the medical probe to define a gap between the first end and the second end of the frame.

18. The lighting apparatus for releasable attachment to a medical probe of claim 15, wherein the frame is continuous and is configured to wrap around the circumference of an ultrasound probe handle.

19. The lighting apparatus for releasable attachment to a medical probe of claim 15, wherein the lighting apparatus is an accessory for a medical probe.
